(19) United States
(12) Patent Application Publication
(10) Pub. No.: US 2007/0255064 A1
(43) Pub. Date: Nov. 1, 2007

(54) Imidazolium-Methyl Sulfites for Use as Starting Compounds for Producing Ionic Liquids

(75) Inventors: Laszlo Szarvas, Ludwigshafen (DE); Klemens Massonne, Bad Durkheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/661,306

(22) PCT Filed: Aug. 3, 2005
(86) PCT No.: PCT/EP05/08412

§ 371 Date: Feb. 23, 2007

(30) Foreign Application Priority Data
Aug. 24, 2004 (DE) 10 2004 041 140.9

(51) Int. Cl. C07D 233/58

(52) U.S. Cl. 548/335.1

(57) ABSTRACT

Imidazolium methylsulfites of the general formula (I) 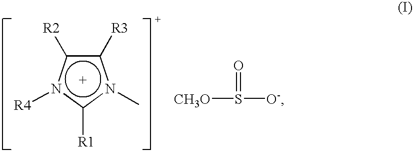 where the radicals have, independently of one another, the following meanings:
R¹ to R³: hydrogen, halogen, hydroxy, amino, aminocarbonyl, sulfo, cyano, nitro;
C₁-C₂₀-alkyl, C₂-C₂₀-alkenyl, C₂-C₂₀-alkynyl, C₁-C₂₀-alkyloxy, C₂-C₂₀-alkenyloxy, C₂-C₂₀-alkynyloxy, C₁-C₂₀-alkylcarbonyl, C₁-C₂₀-alkyloxycarbonyl, C₁-C₂₀-acyloxy, C₁-C₂₀-alkylaminocarbonyl, di(C₁-C₂₀-alkyl)aminocarbonyl, C₁-C₂₀-alkylamino, di(C₁-C₂₀-alkyl)amino, C₁-C₂₀-alkylsulfonyl, C₃-C₈-cycloalkyl, where the alkyl, alkenyl, alkynyl and cycloalkyl groups mentioned can be substituted and/or have their CH₂ groups replaced by oxygen; phenyl, phenoxy, where these can in turn be substituted;
R⁴: sulfo;
C₁-C₂₀-alkyl, C₂-C₂₀-alkenyl, C₂-C₂₀-alkynyl, C₃-C₈-cycloalkyl, where the alkyl, alkenyl, alkynyl and cycloalkyl groups mentioned can be substituted and/or have their CH₂ groups replaced by oxygen; phenyl, where this can be substituted; or
R¹ together with R⁴, and/or R² together with R³, or R⁴ together with R²:
alkylene —(CH₂)ₙ— where n=4, 5 or 6 or butadienylene —CH═CH—CH═CH—, where these can be substituted and/or have the CH₂ groups replaced by oxygen and the CH groups replaced by NR⁵.

The present invention relates to imidazolium methylsulfites of the general formula (I) 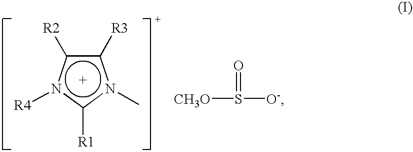
where the radicals have, independently of one another, the following meanings:
R¹ to R³:
hydrogen, halogen, hydroxy, amino, aminocarbonyl, sulfo, cyano, nitro;
C₁-C₂₀-alkyl, C₂-C₂₀-alkenyl, C₂-C₂₀-alkynyl, C₁-C₂₀-alkyloxy, C₂-C₂₀-alkenyloxy, C₂-C₂₀-alkynyloxy, C₁-C₂₀-alkylcarbonyl, C₁-C₂₀-alkyloxycarbonyl, C₁-C₂₀-acyloxy, C₁-C₂₀-alkylaminocarbonyl, di(C₁-C₂₀-alkyl)aminocarbonyl, C₁-C₂₀-alkylamino, di(C₁-C₂₀-alkyl)amino, C₁-C₂₀-alkylsulfonyl, where the alkyl, alkenyl and alkynyl groups mentioned
   can be partially or fully halogenated; and/or
   can bear from one to three of the following groups:
      hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
   can have from one to five of their CH₂ groups replaced by oxygen, with at least one carbon-comprising group being present between any two oxygen heteroatoms;
C₃-C₈-cycloalkyl, where this
   can be partially or fully halogenated; and/or
   can bear from one to three of the following groups:
      hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
   can have from one to three of its CH₂ groups replaced by oxygen, with at least one carbon-comprising group being present between any two oxygen heteroatoms;
phenyl, phenoxy, where these in turn
   can be partially or fully halogenated; and/or
   can bear from one to three of the following groups:
      hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl;
R⁴:
sulfo;
C₁-C₂₀-alkyl, C₂-C₂₀-alkenyl, C₂-C₂₀-alkynyl, where the alkyl, alkenyl and alkynyl groups mentioned
   can be partially or fully halogenated; and/or
   can bear from one to three of the following groups:
      hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
   can have from one to five of their CH₂ groups replaced by oxygen, with at least one carbon-comprising group being present between any two oxygen heteroatoms;
C₃-C₈-cycloalkyl, where this
   can be partially or fully halogenated; and/or
   can bear from one to three of the following groups:
      hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
   can have from one to three of its CH₂ groups replaced by oxygen, with at least one carbon-comprising group being present between any two oxygen heteroatoms;
phenyl, where this
   can be partially or fully halogenated; and/or
   can bear from one to three of the following groups:
      hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; or
R¹ together with R⁴, and/or R² together with R³, or R⁴ together with R²:
alkylene —(CH₂)ₙ— where n=4, 5 or 6, where this
   can be partially or fully halogenated; and/or
   can bear from one to three of the following groups:
      hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
   can have one or two of its CH₂ groups replaced by oxygen or NR⁵, with at least one carbon-comprising group being present between any two heteroatoms;
butadienylene —CH═CH—CH═CH—, which
   can bear from one to three of the following groups:
      hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
   can have one or two of its CH groups replaced by NR⁵, with at least one carbon-comprising group being present between any two heteroatoms;
R⁵:
hydrogen, C₁-C₆-alkyl, C₁-C₆-haloalkyl or C₁-C₆-alkyloxy.

Imidazolium salts are important substances which are used in a wide variety of applications. Thus, they are used, for example, as active ingredients in laundry softeners, in personal hygiene products and cosmetics, as phase transfer catalysts or as electrolyte salts for electronic applications. A further important application area is ionic liquids.

Imidazolium salts are usually prepared by alkylation of the corresponding imidazole.

WO 96/18459 discloses the preparation of halide-free ionic liquids by reaction of a halide of the desired imidazolium, pyridinium or phosphonium cation with a lead salt whose anion represents the anion desired for the ionic liquid and removal of the precipitated lead halide. Disadvantages of the process described are the use of stoichiometric amounts of toxic, generally readily water-soluble lead salts and the formation of stoichiometric amounts of lead halides which have to be disposed of or worked up again.

WO 02/34722 teaches two synthetic routes for preparing 1,2,3-substituted, 1,2,3,4-substituted and 1,2,3,4,5-substituted imidazolium salts: (1) In the first route, the corresponding, substituted imidazole is reacted with an organic halide and the halide ion of the substituted imidazolium halide is replaced by the desired anion by ion exchange. (2) In the second route, the corresponding, substituted imidazole is reacted with an alkyl triflate or a trialkyloxonium salt of the desired anion (e.g. triethyloxonium tetrafluoroborate). Disadvantages of the two methods of preparation are the high costs incurred by the ion exchange which is necessary subsequently and the extraction of the product with an organic solvent which may be associated therewith. A further disadvantage of the preparation via route (1) is the residual content of halide ions which interferes, in particular, when the ionic liquid is used in the electronics industry or in catalysis. A further disadvantage in the preparation via route (2) is that the trialkyloxonium salts used can be obtained only by means of a relatively complicated preparation and both their availability and economic aspects therefore stand in the way of their industrial use.

EP-A 1 182 196 teaches the halide-free preparation of 1,3-substituted imidazolium salts by reaction of the parent 1-substituted imidazole with the corresponding organic disulfate as alkylating agent and subsequent ion exchange with a metal salt comprising the desired anion. Thus, for example, 1-butyl-3-methylimidazolium tetrafluoroborate is prepared by reaction of 1-butylimidazole with dimethyl sulfate, subsequent treatment with sodium tetrafluoroborate and multiple extraction of the product with methylene chloride. Although the process mentioned leads to apparently chloride-free 1,3-substituted imidazolium salts, it has the critical disadvantage of the use of organic disulfate which, owing to its carcinogenic and corrosive nature, represents an increased safety risk and thus requires extensive safety precautions. Furthermore, ion exchange by addition of a metal salt comprising the desired anion is very complicated since the mixture subsequently has to be worked up by extraction using an organic solvent.

In each of the synthetic routes mentioned above, the alkylating agent introduces an anion which subsequently has to be replaced by the desired anion by means of classical anion exchange. This exchange is technically complicated and is generally incomplete, so that the product still contains interfering impurities. This is also true of chloride-free synthetic routes such as those using dialkyl sulfate as alkylating agent, since in this case too, at least small amounts of undesirable sulfate remain in the product after anion exchange.

EP-A 0 291 074 teaches the preparation of quaternary ammonium salts in a two-step reaction by reaction of a tertiary amine with a carbonic diester to form a quaternary ammonium carbonate and mixing of the quaternary ammonium carbonate obtained with an acid to eliminate carbon dioxide and form the quaternary ammonium salt.

EP-A 1 398 318 discloses the halide-free preparation of 1,3-substituted imidazolium salts by reaction of the parent 1-substituted imidazole with a carbonic diester and subsequent reaction of the reaction product obtained with the desired protic acid or a salt thereof to introduce the desired anion.

In contrast to the case of the alkylating agents mentioned further above, the alkylcarbonate anion formed in the alkylation can in principle also be removed completely without classical ion exchange, namely by decomposition into the corresponding alkyl alcohol and gaseous carbon dioxide. However, a disadvantage of the use of dialkyl carbonate as alkylating agent is its relatively low reactivity, so that reaction temperatures of above 100° C. and superatmospheric pressure (e.g. autoclave) are generally necessary.

It was an object of the present invention to find a starting compound for preparing ionic liquids comprising a 1,3-disubstituted imidazolium cation which can be prepared in a simple manner, without use of toxic alkylating agents, using an alkylating agent which can readily be prepared industrially, is readily available and reactive, in high yield and high purity. The high purity should also be able to be achieved without complicated purification. Furthermore, the starting compound to be found should be able to be converted simply and flexibly into the desired ionic liquid having the desired anion, and this should be able to be prepared in high yield and high purity, even without a separate, complicated purification.

We have accordingly found the imidazolium methylsulfites of the general formula (I).

In this formula, the substituents have the following meanings:
halogen:
   fluorine, chlorine, bromine, iodine;
C₁-C₂₀-alkyl which
   can be partially or fully halogenated; and/or
   can bear from one to three of the following groups:
      hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
   can have from one to five of its CH₂ groups replaced by oxygen, with at least one carbon-comprising group being present between any two oxygen heteroatoms;
for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 2-ethylhexyl, 2,4,4-trimethylpentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl, 1-nonadecyl, 1-eicosyl, cyclopentylmethyl, 2-cyclopentylethyl, 3-cyclopentylpropyl, cyclohexylmethyl, 2-cyclohexylethyl, 3-cyclohexylpropyl, benzyl (phenylmethyl), diphenylmethyl (benzhydryl), triphenylmethyl, 1-phenylethyl, 2-phenylethyl, 3-phenylpropyl, α,α-dimethylbenzyl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di(methoxycarbonyl)ethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, methoxymethyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl, 6-ethoxyhexyl, 2-butoxyethyl, 2-isopropoxyethyl, 2-butoxypropyl, 2-octyloxyethyl, 2-methoxyisopropyl, diethoxymethyl, diethoxyethyl, 2-(methoxycarbonyl)ethyl, 2-(ethoxycarbonyl)ethyl, 2-(n-butoxycarbonyl)ethyl, CₙF₍₂ₙ₊₁₎₋ₐHₐ where n is from 1 to 20, 0≦a≦2n+1 (for example CF₃, C₂F₅, CH₂CH₂—C₍ₙ₋₂₎F₂₍ₙ₋₂₎₊₁, C₆F₁₃, C₈F₁₇, C₁₀F₂₁, C₁₂F₂₅), CₙCl₍₂ₙ₊₁₎₋ₐHₐ where n is from 1 to 20, 0≦a≦2n+1, chloromethyl, 2-chloroethyl, trichloromethyl, 1,1-dimethyl-2-chloroethyl, 2,2,2-trifluoroethyl, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-dioxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-dioxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl, 14-ethoxy-5,10-dioxatetradecyl;
   C₂-C₂₀-alkenyl which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example ethenyl (vinyl), propen-1-yl, propen-2-yl, propen-3-yl, 2-phenylethenyl (styryl);
   C₂-C₂₀-alkynyl which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example ethynyl, 1-propynyl, 3-propynyl, 2-phenylethynyl;
   C₁-C₂₀-alkyloxy which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example methoxy, ethoxy, 1-propoxy, 1-butoxy, 1-pentoxy, 1-hexoxy, 2-hydroxyethoxy, 2-methoxyethoxy, 2-ethoxyethoxy, 2-propoxyethoxy, 2-butoxyethoxy, 2-phenoxyethoxy, 5-hydroxy-3-oxapentyloxy, 5-methoxy-3-oxapentyloxy, 5-ethoxy-3-oxapentyloxy, 5-propoxy-3-oxapentyloxy, 5-butoxy-3-oxapentyloxy, 5-phenoxy-3-oxapentyloxy, 8-hydroxy-3,6-dioxahexyloxy, 8-methoxy-3,6-dioxahexyloxy, 8-ethoxy-3,6-dioxahexyloxy, 8-propoxy-3,6-dioxahexyloxy, 8-butoxy-3,6-dioxahexyloxy, 8-phenoxy-3,6-dioxahexyloxy, 3-hydroxypropoxy, 3-methoxypropoxy, 3-ethoxypropoxy, 3-propoxypropoxy, 3-butoxypropoxy, 3-phenoxypropoxy, 7-hydroxy-4-oxaheptyloxy, 7-methoxy-4-oxaheptyloxy, 7-ethoxy-4-oxaheptyloxy, 7-propoxy-4-oxaheptyloxy, 7-butoxy-4-oxaheptyloxy, 7-phenoxy-4-oxaheptyloxy, 11-hydroxy-4,8-dioxaundecyloxy, 11-methoxy-4,8-dioxaundecyloxy, 11-ethoxy-4,8-dioxaundecyloxy, 11-propoxy-4,8-dioxaundecyloxy, 11-butoxy-4,8-dioxaundecyloxy, 11-phenoxy-4,8-dioxaundecyloxy, 4-hydroxybutoxy, 4-methoxybutoxy, 4-ethoxybutoxy, 4-propoxybutoxy, 4-butoxybutoxy, 4-phenoxybutoxy, 9-hydroxy-5-oxanonyloxy, 9-methoxy-5-oxanonyloxy, 9-ethoxy-5-oxanonyloxy, 9-propoxy-5-oxanonyloxy, 9-butoxy-5-oxanonyloxy, 9-phenoxy-5-oxanonyloxy, 14-hydroxy-5,10-dioxatetradecyloxy, 14-methoxy-5,10-dioxatetradecyloxy, 14-ethoxy-5,10-dioxatetradecyloxy, 14-propoxy-5,10-dioxatetradecyloxy, 14-butoxy-5,10-dioxatetradecyloxy, 14-phenoxy-5,10-dioxatetradecyloxy;
   C₂-C₂₀-alkenyloxy which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example prop-2-enyloxy;
   C₂-C₂₀-alkynyloxy which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example prop-2-ynyloxy;
   C₁-C₂₀-alkylcarbonyl which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example acetyl, propionyl, 1-oxobutyl (butyryl), 1-oxopentyl, 1-oxohexyl, 1-oxoheptyl, 1-oxooctyl, 1-oxononyl, 1-oxodecyl, 1-oxoundecyl, 1-oxododecyl, 1-oxotetradecyl, 1-oxohexadecyl, 1-oxooctadecyl, 1-oxoeicosyl, 1,3-dioxobutyl, phenylacetyl, 3-phenylpropionyl, 1-oxo-4-phenylbutyl (4-phenylbutyryl), benzoyl;
   C₁-C₂₀-alkyloxycarbonyl which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example methyloxycarbonyl (CH₃—O—CO—), ethyloxycarbonyl, 1-propyloxycarbonyl, 1-butyloxycarbonyl, 1-pentyloxycarbonyl, 1-hexyloxycarbonyl, 1-heptyloxycarbonyl, 1-octyloxycarbonyl;
   C₁-C₂₀-acyloxy which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-containing group being present between any two oxygen heteroatoms;
for example acetoxy, propionyloxy, 1-oxobutyloxy (butyryloxy), 1-oxopentyloxy, 1-oxohexyloxy, 1-oxoheptyloxy, 1-oxooctyloxy, 1-oxononyloxy, 1-oxodecyloxy, 1-oxoundecyloxy, 1-oxododecyloxy, 1-oxotetradecyloxy, 1-oxohexadecyloxy, 1-oxooctadecyloxy, 1-oxoeicosyloxy, phenylacetoxy, 3-phenylpropionyloxy, 1-oxo-4-phenylbutyloxy (4-phenylbutyryloxy), benzoyloxy;
   C₁-C₂₀-alkylaminocarbonyl, di(C₁-C₂₀-alkyl)aminocarbonyl, which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of their CH₂ groups replaced by oxygen, with at least one carbon-comprising group being present between any two oxygen heteroatoms;
for example N-methylaminocarbonyl, N-ethylaminocarbonyl, N-(1-propyl)aminocarbonyl, N-(2-propyl)aminocarbonyl, N-(1-butyl)aminocarbonyl, N-(2-butyl)aminocarbonyl, N-(2-methyl-1-propyl)aminocarbonyl (N-isobutylaminocarbonyl), N-(2-methyl-2-propyl)aminocarbonyl (N-tert-butylaminocarbonyl), N-(1-pentyl)aminocarbonyl, N-(2-pentyl)aminocarbonyl, N-(3-pentyl)aminocarbonyl, N-(2-methyl-1-butyl)aminocarbonyl, N-(3-methyl-1-butyl)aminocarbonyl, N-(2-methyl-2-butyl)aminocarbonyl, N-(3-methyl-2-butyl)aminocarbonyl, N-(2,2-dimethyl-1-propyl)aminocarbonyl, N-(1-hexyl)aminocarbonyl, N-(2-hexyl)aminocarbonyl, N-(3-hexyl)aminocarbonyl, N-(2-methyl-1-pentyl)aminocarbonyl, N-(3-methyl-1-pentyl)aminocarbonyl, N-(4-methyl-1-pentyl)aminocarbonyl, N-(2-methyl-2-pentyl)aminocarbonyl, N-(3-methyl-2-pentyl)aminocarbonyl, N-(4-methyl-2-pentyl)aminocarbonyl, N-(2-methyl-3-pentyl)aminocarbonyl, N-(3-methyl-3-pentyl)aminocarbonyl, N-(2,2-dimethyl-1-butyl)aminocarbonyl, N-(2,3-dimethyl-1-butyl)aminocarbonyl, N-(3,3-dimethyl-1-butyl)aminocarbonyl, N-(2-ethyl-1-butyl)aminocarbonyl, N-(2,3-dimethyl-2-butyl)aminocarbonyl, N-(3,3-dimethyl-2-butyl)aminocarbonyl, N-(heptyl)aminocarbonyl, N-(octyl)aminocarbonyl, N-(2-ethylhexyl)aminocarbonyl, N-(2,4,4-trimethylpentyl)aminocarbonyl, N-(1,1,3,3-tetramethylbutyl)aminocarbonyl, N-(1-nonyl)aminocarbonyl, N-(1-decyl)aminocarbonyl, N-(1-undecyl)aminocarbonyl, N-(1-dodecyl)aminocarbonyl, N-(1-tetradecyl)aminocarbonyl, N-(1-hexadecyl)aminocarbonyl, N-(1-octadecyl)aminocarbonyl, N-(1-eicosyl)aminocarbonyl, N,N-dimethylaminocarbonyl, N,N-diethylaminocarbonyl, N,N-di(1-propyl)aminocarbonyl, N,N-di(2-propyl)aminocarbonyl, N,N-di(1-butyl)aminocarbonyl, N,N-di(2-butyl)aminocarbonyl, N,N-di(2-methyl-1-propyl)aminocarbonyl (N,N-diisobutylaminocarbonyl), N,N-di(2-methyl-2-propyl)aminocarbonyl (N,N-di-tert-butylaminocarbonyl), N,N-di(1-pentyl)aminocarbonyl, N,N-di(2-pentyl)aminocarbonyl, N,N-di(3-pentyl)aminocarbonyl, N,N-di(2-methyl-1-butyl)aminocarbonyl, N,N-di(3-methyl-1-butyl)aminocarbonyl, N,N-di(2-methyl-2-butyl)aminocarbonyl, N,N-di(3-methyl-2-butyl)aminocarbonyl, N,N-di(2,2-dimethyl-1-propyl)aminocarbonyl, N,N-di(1-hexyl)aminocarbonyl, N,N-di(2-hexyl)aminocarbonyl, N,N-di(3-hexyl)aminocarbonyl, N,N-di(2-methyl-1-pentyl)aminocarbonyl, N,N-di(3-methyl-1-pentyl)aminocarbonyl, N,N-di(4-methyl-1-pentyl)aminocarbonyl, N,N-di(2-methyl-2-pentyl)aminocarbonyl, N,N-di(3-methyl-2-pentyl)aminocarbonyl, N,N-di(4-methyl-2-pentyl)aminocarbonyl, N,N-di(2-methyl-3-pentyl)aminocarbonyl, N,N-di(3-methyl-3-pentyl)aminocarbonyl, N,N-di(2,2-dimethyl-1-butyl)aminocarbonyl, N,N-di(2,3-dimethyl-1-butyl)aminocarbonyl, N,N-di(3,3-dimethyl-1-butyl)aminocarbonyl, N,N-di(2-ethyl-1-butyl)aminocarbonyl, N,N-di(2,3-dimethyl-2-butyl)aminocarbonyl, N,N-di(3,3-dimethyl-2-butyl)aminocarbonyl, N,N-di(1-heptyl)aminocarbonyl, N,N-di(1-octyl)aminocarbonyl, N,N-di(2-ethylhexyl)aminocarbonyl, N,N-di(2,4,4-trimethylpentyl)aminocarbonyl, N,N-di(1,1,3,3-tetramethylbutyl)aminocarbonyl, N,N-di(1-nonyl)aminocarbonyl, N,N-di(1-decyl)aminocarbonyl, N,N-di(1-undecyl)aminocarbonyl, N,N-di(1-dodecyl)aminocarbonyl, N,N-di(1-tetradecyl)aminocarbonyl, N,N-di(1-hexadecyl)aminocarbonyl, N,N-di(1-octadecyl)aminocarbonyl, N,N-di(1-eicosyl)aminocarbonyl, N,N-methylethylaminocarbonyl, N,N-methyl-(1-propyl)aminocarbonyl, N,N-methyl-(1-butyl)aminocarbonyl, N,N-ethyl-(1-propyl)aminocarbonyl, N,N-ethyl-(1-butyl)aminocarbonyl;
   C₁-C₂₀-alkylamino, di(C₁-C₂₀-alkyl)amino which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of their CH₂ groups replaced by oxygen, with at least one carbon-comprising group being present between any two oxygen heteroatoms;
for example N-methylamino, N-ethylamino, N-(1-propyl)amino, N-(2-propyl)amino, N-(1-butyl)amino, N-(2-butyl)amino, N-(2-methyl-1-propyl)amino (N-isobutylamino), N-(2-methyl-2-propyl)amino (N-tert-butylamino), N-(1-pentyl)amino; N-(2-pentyl)amino, N-(3-pentyl)amino, N-(2-methyl-1-butyl)amino, N-(3-methyl-1-butyl)amino, N-(2-methyl-2-butyl)amino, N-(3-methyl-2-butyl)amino, N-(2,2-dimethyl-1-propyl)amino, N-(1-hexyl)amino, N-(2-hexyl)amino, N-(3-hexyl)amino, N-(2-methyl-1-pentyl)amino, N-(3-methyl-1-pentyl)amino, N-(4-methyl-1-pentyl)amino, N-(2-methyl-2-pentyl)amino, N-(3-methyl-2-pentyl)amino, N-(4-methyl-2-pentyl)amino, N-(2-methyl-3-pentyl)amino, N-(3-methyl-3-pentyl)amino, N-(2,2-dimethyl-1-butyl)amino, N-(2,3-dimethyl-1-butyl)amino, N-(3,3-dimethyl-1-butyl)amino, N-(2-ethyl-1-butyl)amino, N-(2,3-dimethyl-2-butyl)amino, N-(3,3-dimethyl-2-butyl)amino, N-(heptyl)amino, N-(octyl)amino, N-(2-ethylhexyl)amino, N-(2,4,4-trimethylpentyl)amino, N-(1,1,3,3-tetramethylbutyl)amino, N-(1-nonyl)amino, N-(1-decyl)amino, N-(1-undecyl)amino, N-(1-dodecyl)amino, N-(1-tetradecyl)amino, N-(1-hexadecyl)amino, N-(1-octadecyl)amino, N-(1-eicosyl)amino, N,N-dimethylamino, N,N-diethylamino, N,N-di(1-propyl)amino, N,N-di(2-propyl)amino, N,N-di(1-butyl)amino, N,N-di(2-butyl)amino, N,N-di(2-methyl-1-propyl)amino, (N,N-diisobutylamino), N,N-di(2-methyl-2-propyl)amino (N,N-di-tert-butylamino), N,N-di(1-pentyl)amino, N,N-di(2-pentyl)amino, N,N-di(3-pentyl)amino, N,N-di(2-methyl-1-butyl)amino, N,N-di(3-methyl-1-butyl)amino, N,N-di(2-methyl-2-butyl)amino, N,N-di(3-methyl-2-butyl)amino, N,N-di(2,2-dimethyl-1-propyl)amino, N,N-di(1-hexyl)amino, N,N-di(2-hexyl)amino, N,N-di(3-hexyl)amino, N,N-di(2-methyl-1-pentyl)amino, N,N-di(3-methyl-1-pentyl)amino, N,N-di(4-methyl-1-pentyl)amino, N,N-di(2-methyl-2-pentyl)amino, N,N-di(3-methyl-2-pentyl)amino, N,N-di(4-methyl-2-pentyl)amino, N,N-di(2-methyl-3-pentyl)amino, N,N-di(3-methyl-3-pentyl)amino, N,N-di(2,2-dimethyl-1-butyl)amino, N,N-di(2,3-dimethyl-1-butyl)amino, N,N-di(3,3-dimethyl-1-butyl)amino, N,N-di(2-ethyl-1-butyl)amino, N,N-di(2,3-dimethyl-2-butyl)amino, N,N-di(3,3-dimethyl-2-butyl)amino, N,N-di(1-heptyl)amino, N,N-di(1-octyl)amino, N,N-di(2-ethylhexyl)amino, N,N-di(2,4,4-trimethylpentyl)amino, N,N-di(1,1,3,3-tetramethylbutyl)amino, N,N-di(1-nonyl)amino, N,N-di(1-decyl)amino, N,N-di(1-undecyl)amino, N,N-di(1-dodecyl)amino, N,N-di(1-tetradecyl)amino, N,N-di(1-hexadecyl)amino, N,N-di(1-octadecyl)amino, N,N-di(1-eicosyl)amino, N,N-methylethylamino, N,N-methyl-(1-propyl)amino, N,N-methyl-(1-butyl)amino, N,N-ethyl-(1-propyl)amino, N,N-ethyl-(1-butyl)amino;
   C₁-C₂₀-alkylsulfonyl which
      can be partially or fully halogenated; and/or
      can bear from one to three of the following groups:
         hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example methylsulfonyl, ethylsulfonyl, 1-propylsulfonyl, 1-butylsulfonyl, 1-pentylsulfonyl, 1-hexylsulfonyl;
   C₃-C₈-cycloalkyl which
      can be partially or fully halogenated; and/or
      can bear from one to three of the following groups:
         hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have from one to five of its CH₂ groups replaced by oxygen, with at least one carbon-comprising group being present between any two oxygen heteroatoms;
for example cyclopropyl, cyclobutyl, cyclopentyl, cyclohexyl, cyclooctyl, cyclododecyl, 1-methylcyclopentyl, 2-methylcyclopentyl, 3-methylcyclopentyl, 1,2-dimethylcyclopentyl, 1,3-dimethylcyclopentyl, 2,2-dimethylcyclopentyl 2,3-dimethylcyclopentyl, 3,3-dimethylcyclopentyl, 1-methylcyclohexyl, 2-methylcyclohexyl, 3-methylcyclohexyl, 4-methylcyclohexyl, 1,2-dimethylcyclohexyl, 1,3-dimethylcyclohexyl, 1,4-dimethylcyclohexyl, 2,2-dimethylcyclohexyl, 2,3-dimethylcyclohexyl, 2,4-dimethylcyclohexyl, 3,3-dimethylcyclohexyl, 3,4-dimethylcyclohexyl, 4,4-dimethylcyclohexyl, 1-methoxycyclopentyl, 2-methoxycyclopentyl, 3-methoxycyclopentyl, 1,2-dimethoxycyclopentyl, 1,3-dimethoxycyclopentyl, 2,2-dimethoxycyclopentyl, 2,3-dimethoxycyclopentyl, 3,3-dimethoxycyclopentyl, 1-methoxycyclohexyl, 2-methoxycyclohexyl, 3-methoxycyclohexyl, 4-methoxycyclohexyl, 1,2-dimethoxycyclohexyl, 1,3-dimethoxycyclohexyl, 1,4-dimethoxycyclohexyl, 2,2-dimethoxycyclohexyl, 2,3-dimethoxycyclohexyl, 2,4-dimethoxycyclohexyl, 3,3-dimethoxycyclohexyl, 3,4-dimethoxycyclohexyl, 4,4-dimethoxycyclohexyl, dichlorocyclopentyl, chlorocyclohexyl, dichlorocyclohexyl, 2,3,4,5,6-pentachlorocyclohexyl, CₙF₍₂ₙ₋₁₎₋ₐHₐ where n is from 3 to 8, 0≦a≦2n−1, CₙCl₍₂ₙ₋₁₎₋ₐHₐ where n is from 3 to 8, 0≦a≦n−1, 1,3-dioxolan-2-yl, 1,3-dioxan-2-yl, 2-methyl-1,3-dioxolan-2-yl, 4-methyl-1,3-dioxolan-2-yl, 2-methyl-1,3-dioxan-2-yl, 4-methyl-1,3-dioxan-2-yl;
   phenyl, phenoxy, which
      can be partially or fully halogenated; and/or
      can bear from one to three of the following groups:
         hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl;
for example phenyl, 2-methylphenyl (2-tolyl), 3-methylphenyl (3-tolyl), 4-methylphenyl (4-tolyl), 2-ethylphenyl, 3-ethylphenyl, 4-ethylphenyl, 2-methoxyphenyl, 3-methoxyphenyl, 4-methoxyphenyl, 2-sulfophenyl (o-tosyl), 3-sulfophenyl (m-tosyl), 4-sulfophenyl (p-tosyl), phenoxy, 2-methylphenoxy, 3-methylphenoxy, 4-methylphenoxy, 2-ethylphenoxy, 3-ethylphenoxy, 4-ethylphenoxy, 2-methoxyphenoxy, 3-methoxyphenoxy, 4-methoxyphenoxy;
   alkylene —(CH₂)ₙ— where n=4, 5 or 6, which
      can be partially or fully halogenated; and/or
      can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups:
         hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have one or two of its CH₂ groups replaced by oxygen or NR⁵, with at least one carbon-comprising group being present between any two heteroatoms;
for example 1,4-butylene, 1,5-pentylene, 1,6-hexylene, CₙF₂ₙ₋ₐHₐ where n is from 4 to 6, 0≦a≦2n;
   butadienylene —CH═CH—CH═CH— which
      can bear from one to three of the following groups:
         hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or
      can have one or two of its CH groups replaced by NR⁵, with at least one carbon-comprising group being present between any two heteroatoms;
for example butadienylene.

The radicals R¹ to R³ in the imidazolium methylsulfites (I) of the invention preferably have the following meanings
hydrogen, fluorine, chlorine, aminocarbonyl, sulfo, cyano;
C₁-C₂₀-alkyl, C₁-C₂₀-alkyloxy, C₁-C₂₀-alkylcarbonyl, C₁-C₂₀-alkyloxycarbonyl, C₁-C₂₀-acyloxy, C₁-C₂₀-alkylaminocarbonyl, di(C₁-C₂₀-alkyl)aminocarbonyl, C₁-C₂₀-alkylamino, di(C₁-C₂₀-alkyl)amino, where the alkyl groups mentioned
   can be partially or fully fluorinated or chlorinated; and/or
   can bear from one to three of the following groups:
      hydroxy, sulfo, cyano, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully fluorinated or chlorinated and/or can bear from one to three of the following groups: hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl; and/or
   can have from one to five of their CH₂ groups replaced by oxygen, with at least one carbon-comprising group being present between any two oxygen heteroatoms;
C₃-C₈-cycloalkyl, where this
   can be partially or fully fluorinated or chlorinated; and/or
   can bear from one to three of the following groups:
      hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl; and/or
   can have from one to three of its CH₂ groups replaced by oxygen, with at least one carbon-comprising group being present between any two oxygen heteroatoms;
phenyl, phenoxy, where these in turn
   can be partially or fully fluorinated or chlorinated; and/or
   can bear from one to three of the following groups:
      hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl.

The radicals R¹ to R³ in the imidazolium methylsulfites (I) of the invention particularly preferably have the following meanings
hydrogen, fluorine, chlorine;
C₁-C₆-alkyl, C₁-C₆-alkyloxy, C₅-C₆-cycloalkyl, where the alkyl and cycloalkyl groups mentioned
   can be partially or fully fluorinated or chlorinated; and/or
   can have one or two of their CH₂ groups replaced by oxygen, with at least one carbon-comprising group being present between any two oxygen heteroatoms;
phenyl, where this
   can be partially or fully fluorinated or chlorinated; and/or
   can bear from one to three of the following groups:
      C₁-C₆-alkyl, C₁-C₆-haloalkyl, C₁-C₆-alkyloxy.

Examples of particularly preferred radicals R¹ to R³ are: hydrogen, fluorine, chlorine, methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, methoxymethyl, 2-methoxyethyl, 2-methoxypropyl, 3-methoxypropyl, 4-methoxybutyl, 2-ethoxyethyl, 2-ethoxypropyl, 3-ethoxypropyl, 4-ethoxybutyl, 2-butoxyethyl, 2-isopropoxyethyl, 2-methoxyisopropyl, diethoxymethyl, diethoxyethyl, CₙF₍₂ₙ₊₁₎₋ₐHₐ where n is from 1 to 6, 0≦a≦2n+1 (for example CF₃, C₂F₅, CH₂CH₂—C₍ₙ₋₂₎F₂₍ₙ₋₂₎₊₁, C₆F₁₃), CₙCl₍₂ₙ₊₁₎₋ₐHₐ where n is from 1 to 6, 0≦a≦2n+1, chloromethyl, 2-chloroethyl, trichloromethyl, 1,1-dimethyl-2-chloroethyl, 2,2,2-trifluoroethyl, 5-methoxy-3-oxapentyl, methoxy, ethoxy, 1-propoxy, 1-butoxy, 1-pentoxy, 1-hexoxy, 2-methoxyethoxy, 2-ethoxyethoxy, 2-propoxyethoxy, 2-butoxyethoxy, 5-methoxy-3-oxapentyloxy, 3-methoxypropoxy, 3-ethoxypropoxy, 4-methoxybutoxy, 4-ethoxybutoxy, cyclopentyl, cyclohexyl, 2,3,4,5,6-pentachlorocyclohexyl, CₙF₍₂ₙ₋₁₎₋ₐHₐ where n is from 5 to 6, 0≦a≦2n−1, CₙCl₍₂ₙ₋₁₎₋ₐHₐ where n is from 5 to 6, 0≦a≦n−1, 1,3-dioxolan-2-yl, 1,3-dioxan-2-yl, phenyl, 2-methylphenyl (2-tolyl), 3-methylphenyl (3-tolyl), 4-methylphenyl (4-tolyl), 2-ethylphenyl, 3-ethylphenyl, 4-ethylphenyl, 2-methoxyphenyl, 3-methoxyphenyl, 4-methoxyphenyl.

The radicals R¹ to R³ in the imidazolium methylsulfites (I) of the invention are very particularly preferably hydrogen, methyl, ethyl, 1-propyl, 1-butyl, 1-pentyl, 1-hexyl, 1-octyl, 1-decyl, 1-dodecyl, 1-pentadecyl, in particular hydrogen, methyl, ethyl, 1-butyl, 1-hexyl, 1-octyl, 1-decyl.

The radical R⁴ in the imidazolium methylsulfites (I) of the invention preferably has the following meanings
sulfo;
C₁-C₂₀-alkyl, where this
   can be partially or fully fluorinated or chlorinated; and/or
   can bear from one to three of the following groups:
      hydroxy, sulfo, cyano, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully fluorinated or chlorinated and/or can bear from one to three of the following groups: hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl; and/or
   can have from one to five of its CH₂ groups replaced by oxygen, with at least one carbon-comprising group being present between any two oxygen heteroatoms;
C₃-C₈-cycloalkyl, where this
   can be partially or fully fluorinated or chlorinated; and/or
   can bear from one to three of the following groups:
      hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl; and/or
   can have from one to three of its CH₂ groups replaced by oxygen, with at least one carbon-comprising group being present between any two oxygen heteroatoms.

The radical R⁴ in the imidazolium methylsulfites (I) of the invention particularly preferably has the following meanings:
sulfo;
C₁-C₆-alkyl, C₅-C₆-cycloalkyl, where the alkyl and cycloalkyl groups mentioned
   can be partially or fully fluorinated or chlorinated; and/or
   can have one or two of their CH₂ groups replaced by oxygen, with at least one carbon-comprising group being present between any two oxygen heteroatoms.

Examples of particularly preferred radicals R⁴ are: sulfo, methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, CₙF₍₂ₙ₊₁₎₋ₐHₐ where n is from 1 to 6, 0≦a≦2n+1 (for example CF₃, C₂F₅, CH₂CH₂—C₍ₙ₋₂₎F₂₍ₙ₋₂₎₊₁, C₆F₁₃), CₙCl₍₂ₙ₊₁₎₋ₐHₐ where n is from 1 to 6, 0≦a≦2n+1, chloromethyl, 2-chloroethyl, trichloromethyl, 1,1-dimethyl-2-chloroethyl, 2,2,2-trifluoroethyl, cyclopentyl, cyclohexyl, 2,3,4,5,6-pentachlorocyclohexyl, CₙF₍₂ₙ₋₁₎₋ₐHₐ where n is from 5 to 6, 0≦a≦2n−1, CₙCl₍₂ₙ₋₁₎₋ₐHₐ where n is from 5 to 6, 0≦a≦n−1, 1,3-dioxolan-2-yl, 1,3-dioxan-2-yl.

The radical R⁴ in the imidazolium methylsulfites (I) of the invention likewise preferably has the following meanings
C₁-C₂₀-alkyl, where this
   can be partially or fully fluorinated or chlorinated; and/or
   can bear from one to three of the following groups:
      hydroxy, sulfo, cyano, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully fluorinated or chlorinated and/or can bear from one to three of the following groups: hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl; and/or
   can have from one to five of its CH₂ groups replaced by oxygen, with at least one carbon-comprising group being present between any two oxygen heteroatoms;
C₃-C₈-cycloalkyl, where this
   can be partially or fully fluorinated or chlorinated; and/or
   can bear from one to three of the following groups:
      hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl; and/or
   can have from one to three of its CH₂ groups replaced by oxygen, with at least one carbon-comprising group being present between any two oxygen heteroatoms.

The radical R⁴ in the imidazolium methylsulfites (I) of the invention particularly preferably has the following meanings:
C₁-C₆-alkyl, C₅-C₆-cycloalkyl, where the alkyl and cycloalkyl groups mentioned
   can be partially or fully fluorinated or chlorinated; and/or
   can have one or two of their CH₂ groups replaced by oxygen, with at least one carbon-comprising group being present between any two oxygen heteroatoms.

Examples of particularly preferred radicals R⁴ are: methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, CₙF₍₂ₙ₊₁₎₋ₐHₐ where n is from 1 to 6, 0≦a≦2n+1 (for example CF₃, C₂F₅, CH₂CH₂—C₍ₙ₋₂₎F₂₍ₙ₋₂₎₊₁, C₆F₁₃), CₙCl₍₂ₙ₊₁₎₋ₐHₐ where n is from 1 to 6, 0≦a≦2n+1, chloromethyl, 2-chloroethyl, trichloromethyl, 1,1-dimethyl-2-chloroethyl, 2,2,2-trifluoroethyl, cyclopentyl, cyclohexyl, 2,3,4,5,6-pentachlorocyclohexyl, CₙF₍₂ₙ₋₁₎₋ₐHₐ where n is from 5 to 6, 0≦a≦2n−1, CₙCl₍₂ₙ₋₁₎₋ₐHₐ where n is from 5 to 6, 0≦a≦n−1, 1,3-dioxolan-2-yl, 1,3-dioxan-2-yl.

The radical R⁴ in the imidazolium methylsulfites (I) of the invention is very particularly preferably methyl, ethyl, 1-propyl, 1-butyl, 1-pentyl, 1-hexyl, 1-octyl, 1-decyl, 1-dodecyl, 1-pentadecyl, in particular methyl, ethyl, 1-butyl, 1-hexyl, 1-octyl, 1-decyl.

Very particularly preferred imidazolium methylsulfites (I) are: N,N′-dimethylimidazolium methylsulfite, N,N′-ethylmethylimidazolium methylsulfite, N,N′-(1-propyl)methylimidazolium methylsulfite, N,N′-(1-butyl)methylimidazolium methylsulfite, N,N′-(1-pentyl)methylimidazolium methylsulfite, N,N′-(1-hexyl)methylimidazolium methylsulfite, N,N′-(1-octyl)methylimidazolium methylsulfite, N,N′-(1-decyl)methylimidazolium methylsulfite, N,N′-(1-dodecyl)methylimidazolium methylsulfite, N,N′-(1-pentadecyl)methylimidazolium methylsulfite.

The imidazolium methylsulfites (I) of the invention can be prepared by reacting the corresponding imidazole of the general formula (II) 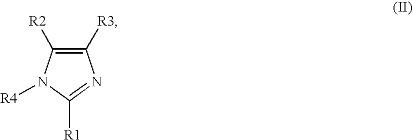
where the radicals R¹ to R⁴ have the meanings given for the formula (I), with dimethyl sulfite. For this purpose, the two reactants (starting materials) are combined. The manner and order of addition of the two starting materials is unimportant in the preparative process. Thus, for example, it is possible to introduce the imidazole (II) and the dimethyl sulfite into the reaction apparatus in succession in any order or simultaneously. It is also possible to place one of the two starting materials in the reaction apparatus first and add the other starting material dropwise over a period of from a few minutes to a number of hours.

Reaction apparatuses which can be used in the preparative process are in principle all reaction apparatuses which are suitable for a reaction in the liquid phase. These are, in particular, reaction apparatuses which make appropriate mixing of the liquid starting materials possible, for example stirred vessels.

The molar ratio of dimethyl sulfite to the imidazole (II) in the preparative process is generally from 0.9 to 1.5, preferably from 0.9 to 1.2, particularly preferably from 0.9 to 1.1 and very particularly preferably from 0.95 to 1.05.

The reaction between the imidazole (II) and the dimethyl sulfite in the preparative process is generally carried out at a temperature of from 10 to 150° C. However, it has been found, according to the invention, that the content of the isomeric imidazolium methanesulfonate generally also increases with increasing reaction temperature due to the thermally induced isomerization of the methylsulfite anion. To obtain particularly pure imidazolium methylsulfite (I), preference is therefore given to carrying out the reaction at a temperature of ≦100° C. The reaction is thus preferably carried out at a temperature of from 10 to 100° C., particularly preferably from 20 to 100° C., very particularly preferably from 30 to 90° C. and in particular from 50 to 80° C. The reaction is generally carried out at a pressure of from 0.05 to 2 MPa abs, preferably from 0.09 to 0.5 MPa abs, particularly preferably from 0.09 to 0.2 MPa abs and very particularly preferably from 0.095 to 0.12 MPa abs.

The time required for the reaction is dependent first and foremost on the chemical nature of the starting material (reactivity of the imidazole (II)) and the reaction temperature selected. It can be determined, for instance, by means of preliminary experiments in which, for example, the reaction kinetics are determined, the temperature curve of the exothermic reaction is measured and/or the concentration of the starting materials and product are determined by analysis. In general, the time required is in the range from a few minutes to one day, generally of the order of from 0.1 to 24 hours, preferably of the order of from 0.1 to 10 hours.

The reaction of the imidazole (II) with the dimethyl sulfite forms the imidazolium methylsulfite (I) in a stoichiometric amount according to the following reaction equation, 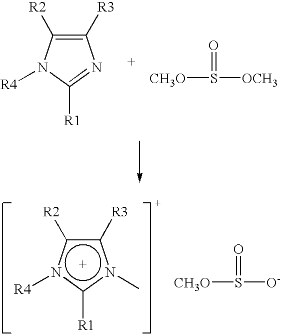
where the radicals R¹ to R⁴ have the meanings given for the formula (I). In general, the imidazolium methylsulfite (I) is obtained in relatively pure form and can, depending on the desired purity, be isolated directly or be subjected to a subsequent purification step as described below.

The preparative process can also be carried out using an inorganic or organic solvent which should be inert toward the two starting materials imidazole (II) and dimethyl sulfite and the product imidazolium methylsulfite (I) under the reaction conditions. Solvents which are suitable in principle are, for example, aliphatic, araliphatic or aromatic hydrocarbons, alcohols, ethers, halogenated hydrocarbons, tetrahydrofuran or acetonitrile. If solvents are used, these preferably have a relatively low polarity, in particular compared to the solvents described in the prior art, for instance acetonitrile and alcohol in JP 2001-322,970 or methanol, chloroform and nitrobenzene in DE 228 247. This relatively low polarity leads to the imidazolium methylsulfite (I) formed during the reaction forming a separate solid or liquid phase and unreacted starting material or possible by-products, for example, therefore remaining preferentially in the solvent phase.

In this context, it has been found, according to the invention, that a particularly pure imidazolium methylsulfite (I) can be obtained in high yield in the preparative process when the reaction is carried out
(i) in the presence of a solvent selected from the group consisting of aromatic hydrocarbons having from 6 to 10 carbon atoms, symmetrical or unsymmetrical dialkyl ethers having a-total of from 5 to 10 carbon atoms, cycloalkanes having from 5 to 8 carbon atoms and C₅-C₁₀-alkanes; and
(ii) at a temperature of from 10 to 100° C.

Aromatic hydrocarbons having from 6 to 10 carbon atoms which are used are generally unsubstituted benzene or benzene substituted by C₁-C₄-alkyl, —CH═CH—CH═CH—, 1,4-butylene, —O—CH₂—CH₂—CH₂— and also monohydroxyalkylbenzenes or monoalkoxyalkylbenzenes having a number of carbon atoms in the specified range. Examples of suitable hydrocarbons having from 6 to 10 carbon atoms are benzene, toluene, ethylbenzene, 1-propylbenzene, 2-propylbenzene, 1-butylbenzene, 2-butylbenzene, tert-butylbenzene, xylene (o-, m-, p-), methylethylbenzene (o-, m-, p-), diethylbenzene (o-, m-, p-), trimethylbenzene (vic-, sym-, asym-), cresol (o-, m-, p-), ethylphenol (o-, m-, p-), 1,2,3,4-tetrahydronaphthalene.

Symmetrical or unsymmetrical dialkyl ethers having a total of from 5 to 10 carbon atoms which are used are generally dialkyl ethers having unbranched or branched alkyl groups, with at least one alkyl group being a C₃-C₉-alkyl group. The number of carbon atoms in the other alkyl group is determined by the specified total number of carbon atoms. Examples of suitable symmetrical or unsymmetrical dialkyl ethers having a total of from 5 to 10 carbon atoms are diisopropyl ether, methyl tert-butyl ether, di-n-butyl ether and diethylene glycol dimethyl ether.

Cycloalkanes having from 5 to 8 carbon atoms which are used are generally unsubstituted or C₁-C₃-alkyl-substituted cycloalkanes. Examples of suitable cycloalkanes having from 5 to 8 carbon atoms are cyclopentane, methylcyclopentane, cyclohexane, methylcyclohexane, cycloheptane, cyclooctane.

C₅-C₁₀-Alkanes used are generally unbranched or branched alkanes. Examples of suitable C₅-C₁₀-alkanes are n-pentane, 2-methylbutane (isopentane), 2,2-dimethylpropane, n-hexane, 2-methylpentane, 3-methylpentane, 2,3-dimethylbutane, 2,2-dimethylbutane, n-heptane, isomeric heptanes, n-octane, isomeric octanes, n-nonane, isomeric nonanes, n-decane, isomeric decanes.

It is naturally also possible to use mixtures of various solvents.

In the preferred preparative process, preference is given to using toluene, xylene, ethylbenzene, diethylbenzene, methyl tert-butyl ether, cyclohexane, hexane, heptane or octane as solvent.

The amount of solvent to be used in the preferred preparative process is generally from 10 to 1000% by weight, preferably from 20 to 500% by weight and particularly preferably from 20 to 200% by weight, based on the amount of the imidazole (II) used.

In the preparative process using a solvent, too, the manner and order of addition of the individual starting materials and the solvent is unimportant. Thus, it is possible, for example, to introduce the imidazole (II), the dimethyl sulfite and the solvent into the reaction apparatus in succession in any order or simultaneously. It is also possible to admix the imidazole (II) and/or the dimethyl sulfite with part or the total amount of solvent and only then combine the two solvent-containing starting materials. Furthermore, it is also possible to place one of the two starting materials in the reaction apparatus first and add the other starting material dropwise over a period of from a few minutes to a number of hours, with at least one of the starting materials being diluted with the solvent.

If the preparative process is carried out in the presence of the preferred solvents selected from the abovementioned group consisting of aromatic hydrocarbons having from 6 to 10 carbon atoms, symmetrical or unsymmetrical dialkyl ethers having a total of from 5 to 10 carbon atoms, cycloalkanes having from 5 to 8 carbon atoms and C₅-C₁₀-alkanes, phase separation occurs after mixing of the reaction mixture is stopped after the reaction is complete. Depending on the type of reaction apparatus, it can be advantageous to carry out the settling of the two phases in this apparatus or in a separate settling vessel. After the two phases have settled, the liquid or solid phase of the imidazolium methylsulfite (I) obtained is separated off. In general, the phase of the imidazolium methylsulfite (I) is located at the bottom and the solvent phase is located at the top. The solvent separated off can generally be recirculated and reused as solvent for the reaction in question. It may be advisable to employ measures to prevent accumulation of any by-products in the solvent. Possible measures are, for example, (i) discharge of a small part of the solvent and replacement of this by fresh solvent or (ii) distillation of at least a small part of the solvent with subsequent recirculation.

Depending on the desired purity of the imidazolium methylsulfite (I), it can be advantageous to subject the crude product obtained, whether it is from the preparation without use of a solvent or from the preparation with use of a solvent, to a subsequent purification step. If the phase of the imidazolium methylsulfite (I) is liquid at the working temperature, this can be shaken with a suitable solvent in which the imidazolium methylsulfite (I) is insoluble or only very sparingly soluble. Suitable solvents for this purpose are, for example, the solvents mentioned as preferred for the reaction or esters such as ethyl acetate. If the phase of the imidazolium methylsulfite (I) is solid at the working temperature, it can, for example, be washed with a suitable solvent in which the imidazolium methylsulfite (I) is insoluble or only very sparingly soluble. Suitable solvents for this purpose are, for example, likewise the solvents mentioned as preferred for the reaction or esters such as ethyl acetate. Furthermore, the solid imidazolium methylsulfite (I) can also be recrystallized from a suitable solvent. Suitable solvents for this purpose are solvents in which the imidazolium methylsulfite (I) dissolves, for example alcohols, acetonitrile, tetrahydrofuran or nitrobenzene.

Depending on the further use of the purified or unpurified imidazolium methylsulfite (I), it can be advantageous to dry it beforehand. If drying is carried out, it is preferably carried out under particularly mild temperature conditions under reduced pressure in order to prevent decomposition of the imidazolium methylsulfite (I) and, in particular, isomerization to the imidazolium methanesulfonate.

The preparative process can be carried out batchwise, semicontinuously or continuously. When it is carried out batchwise, the starting materials and, if appropriate, the solvent are combined and the reaction is carried out at the desired temperature. After the reaction is complete, the reaction mixture is worked up as described. When it is carried out continuously, the two starting materials are slowly fed into the reaction apparatus for them to react at the desired temperature. If a solvent is employed, this is added together with one of the two starting materials, divided over the two starting materials or separately. The reaction mixture is taken off continuously in an amount corresponding to the amounts of starting materials and solvent fed in and is worked up as described. The work-up itself can likewise be carried out continuously. In the case of the semicontinuous variants, at least one of the two starting materials is slowly introduced at the desired temperature, with the reaction generally occurring in parallel with the addition. After the desired amount(s) has/have been added, the reaction mixture is generally left to react further for a particular time and is subsequently worked up as described.

The imidazolium methylsulfites (I) of the invention can be used for preparing imidazolium salts of a variety of anions. One anion of industrial relevance is the hydrogensulfite anion. Thus, the imidazolium methylsulfites (I) of the invention are, in a preferred use, employed as starting materials in the preparation of ionic liquids, in which case the imidazolium methylsulfite (I) is reacted with water to liberate methanol and form the imidazolium hydrogensulfite. Reaction apparatuses which can be used are in principle all reaction apparatuses which are suitable for a reaction in the liquid phase. These are, in particular, reaction apparatuses which make appropriate mixing of the liquid starting materials possible, for example stirred vessels. The molar ratio of water to the imidazolium methylsulfite (I) is generally from 0.9 to 1.5, preferably from 0.95 to 1.2, particularly preferably from 0.95 to 1.1 and very particularly preferably from 0.99 to 1.05. The reaction is generally carried out at a temperature of from 10 to 80° C., preferably from 10 to 60° C. and particularly preferably from 20 to 40° C. The pressure is generally from 0.05 to 2 MPa abs, preferably from 0.09 to 0.5 MPa abs and particularly preferably from 0.095 to 0.12 MPa abs. The time required for the reaction is generally from a few minutes to a number of hours, preferably from 0.1 to 5 hours, and can, for example, be determined over the course of the reaction (pH, concentration of the methylsulfite anion). After the reaction is complete, the methanol formed and any excess water present are generally taken off under reduced pressure at a temperature of from 10 to 80° C., preferably from 10 to 60° C. The product obtained can be washed with solvents in which the imidazolium hydrogensulfite is insoluble or only very sparingly soluble, for example an aromatic hydrocarbon having from 6 to 10 carbon atoms, a symmetrical or unsymmetrical dialkyl ether having a total of from 5 to 10 carbon atoms, a cycloalkane having from 5 to 8 carbon atoms or a C₅-C₁₀-alkane. Furthermore, it is also possible to recrystallize the product from a solvent in which the imidazolium hydrogensulfite dissolves, for example an alcohol, acetonitrile, tetrahydrofuran or nitrobenzene. The product is generally dried under reduced pressure.

Further, industrially relevant anions can likewise be prepared in a simple way from the imidazolium methylsulfites (I) of the invention. Thus, in another preferred use, the imidazolium methylsulfites (I) of the invention are employed as starting materials in the preparation of ionic liquids, in which case the imidazolium methylsulfite (I) is reacted with an inorganic or organic protic acid having a pKₐ of from 1.8 to 14, measured at 25° C. in aqueous solution, to liberate methanol and sulfur dioxide and form the imidazolium salt of the corresponding partially or fully deprotonated acid anion. The pKₐ of the inorganic or organic protic acid to be used is preferably from 1.8 to 10, particularly preferably from 2 to 10 and very particularly preferably from 3 to 10, measured at 25° C. in aqueous solution. Reaction apparatuses which can be used are in principle all reaction apparatuses which are suitable for a reaction in the liquid phase. These are, in particular, reaction apparatuses which make appropriate mixing of the liquid starting materials possible, for example stirred vessels. The molar ratio of the inorganic or organic protic acid to the imidazolium methylsulfite (I) is generally from 0.9 to 1.5, preferably from 0.95 to 1.1, particularly preferably from 0.95 to 1.05 and very particularly preferably from 0.99 to 1.02. The reaction is generally carried out at a temperature of from 10 to 80° C., preferably from 10 to 60° C. and particularly preferably from 20 to 40° C. The pressure is generally from 0.05 to 2 MPa abs, preferably from 0.09 to 0.5 MPa abs and particularly preferably from 0.095 to 0.12 MPa abs. The time required for the reaction is generally from a few minutes to a number of hours, preferably from 0.1 to 5 hours, and can, for example, be determined over the course of the reaction (pH, concentration of the methylsulfite anion). After the reaction is complete, any excess acid present is generally neutralized by means of a base, for example sodium hydroxide, and the product is subsequently washed with a solvent in which the imidazolium salt does not dissolve, for example an alcohol, acetonitrile, tetrahydrofuran or nitrobenzene. The product is generally dried under reduced pressure.

The imidazolium methylsulfite (I) of the invention is preferably used for preparing an imidazolium salt in which the partially or fully deprotonated anion is
fluoride; hexafluorophosphate; hexafluoroarsenate; hexafluoroantimonate; trifluoroarsenate; nitrite; nitrate; sulfate; hydrogensulfate; carbonate; hydrogencarbonate; phosphate; hydrogenphosphate; dihydrogenphosphate, vinylphosphonate, dicyanamide, bis(pentafluoroethyl)phosphinate, tris(pentafluoroethyl)trifluorophosphate, tris(heptafluoropropyl)trifluorophosphate, bis[oxalato(2-)]borate, bis[salicylato(2-)]borate, bis[1,2-benzenediolato(2-)-O,O′]borate, tetracyanoborate, tetracarbonylcobaltate;
tetrasubstituted borate of the general formula (Va) [BR^{a}R^{b}R^{c}R^{d}]⁻, where R^{a} to R^{d} are each, independently of one another, fluorine or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;
organic sulfonate of the general formula (Vb) [R^{e}—SO₃]⁻, where R^{e} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;
carboxylate of the general formula (Vc) [R^{f}—COO]⁻, where R^{f} is hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;
(fluoroalkyl)fluorophosphate of the general formula (Vd) [PFₓ(C_{y}F_{2y+1-z}H_{z})₆₋ₓ]⁻, where 1≦x≦6, 1≦y≦8 and 0≦z≦2y+1;
imide of the general formulae (Ve) [R^{g}—SO₂—N—SO₂—R^{h}]⁻, (Vf) [Rⁱ—SO₂—N—CO—R^{j}]⁻ or (IVg) [R^{k}—CO—N—CO—R^{l}]⁻, where R^{g} to R^{l} are each, independently of one another, hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;
methide of the general formula (Vh) 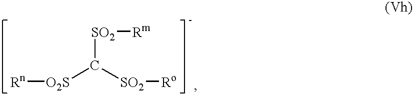
   where R^{m} to R^{o} are, independently of one another, hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;
organic sulfate of the general formula (Vi) [R^{p}O—SO₃]⁻, where R^{p} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;
halometalate of the general formula (Vj) [M_{q}Halᵣ]^{s-}, where M is a metal and Hal is fluorine, chlorine, bromine or iodine, q and r are positive integers and indicate the stoichiometry of the complex and s is a positive integer and indicates the charge on the complex; or
sulfide, hydrogensulfide, hydrogenpolysulfide of the general formula (Vk) [HSᵥ]⁻, polysulfide of the general formula (Vm) [Sᵥ]²⁻, where v is a positive integer from 2 to 10, thiolate of the general formula (Vn) [R^{s}S]⁻, where R^{s} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen.

Possible heteroatoms are in principle all heteroatoms which are able to formally replace a —CH₂— group, a —CH═ group, a C≡ group or a ═C═ group. If the carbon-comprising radical comprises heteroatoms, then preference is given to oxygen, nitrogen, sulfur, phosphorus and silicon. Preferred groups are, in particular, —O—, —S—, —SO—, —SO₂—, —NR—, —N═, —PR—, —PR₂ and —SiR₂—, where the radicals R are the remaining part of the carbon-comprising radical.

Possible functional groups are in principle all functional groups which can be bound to a carbon atom or a heteroatom. Examples of suitable groups are —OH (hydroxy), ═O (in particular as a carbonyl group), —NH₂ (amino), ═NH (imino), —COOH (carboxy), —CONH₂ (carboxamide) and —CN (cyano). Functional groups and heteroatoms can also be directly adjacent, so that combinations of a plurality of adjacent atoms, e.g. —O— (ether), —S— (thioether), —COO— (ester), —CONH— (secondary amide) or —CONR— (tertiary amide), are also encompassed.

As halogens, mention may be made of fluorine, chlorine, bromine and iodine.

Carbon-comprising organic, saturated or unsaturated, acyclic or cyclic, aliphatic, aromatic or araliphatic radicals having from 1 to 30 carbon atoms as the radicals R^{a} to R^{d} in the tetra-substituted borate (Va), the radical R^{e} in the organic sulfonate (Vb), the radical R^{f} in the carboxylate (Vc), the radicals R^{g} to R^{l} in the imides (Ve), (Vf) and (Vg), the radicals R^{m} to R^{o} in the methide (Vh), the radical R^{p} in the organic sulfate (Vi) and the radical R^{s} in the thiolate (Vn) are preferably, independently of one another,
C₁-C₃₀-alkyl and their aryl-, heteroaryl-, cycloalkyl-, halogen-, hydroxy-, amino-, carboxy-, formyl-, —O—, —CO—, —CO—O— or —CO—N<-substituted components, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl, hexacosyl, heptacosyl, octacosyl, nonacosyl, triacontyl, phenylmethyl (benzyl), diphenylmethyl, triphenylmethyl, 2-phenylethyl, 3-phenylpropyl, cyclopentylmethyl, 2-cyclopentylethyl, 3-cyclopentylpropyl, cyclohexylmethyl, 2-cyclohexylethyl, 3-cyclohexylpropyl, methoxy, ethoxy, formyl, acetyl or CₙF_{2(n-a)+(1-b)}H_{2a+b} where n≦30, 0≦a≦n and b=0 or 1 (for example CF₃, C₂F₅, CH₂CH₂—C₍ₙ₋₂₎F₂₍ₙ₋₂₎₊₁, C₆F₁₃, C₈F₁₇, C₁₂F₂₅);
C₃-C₁₂-cycloalkyl and their aryl-, heteroaryl-, cycloalkyl-, halogen-, hydroxy-, amino-, carboxy-, formyl-, —O—, —CO— or —CO—O-substituted components, for example cyclopentyl, 2-methyl-1-cyclopentyl, 3-methyl-1-cyclopentyl, cyclohexyl, 2-methyl-1-cyclohexyl, 3-methyl-1-cyclohexyl, 4-methyl-1-cyclohexyl or CₙF_{2(n-a)(1-b)}H_{2a-b} where n≦30, 0≦a≦n and b=0 or 1;
C₂-C₃₀-alkenyl and their aryl-, heteroaryl-, cycloalkyl-, halogen-, hydroxy-, amino-, carboxy-, formyl-, —O—, —CO— or —CO—O-substituted components, for example 2-propenyl, 3-butenyl, cis-2-butenyl, trans-2-butenyl or CₙF_{2(n-a)-(1-b)}H_{2a-b} where n≦30, 0≦a≦n and b=0 or 1;
C₃-C₁₂-cycloalkenyl and their aryl-, heteroaryl-, cycloalkyl-, halogen-, hydroxy-, amino-, carboxy-, formyl-, —O—, —CO— or —CO—O-substituted components, for example 3-cyclopentenyl, 2-cyclohexenyl, 3-cyclohexenyl, 2,5-cyclohexadienyl or CₙF_{2(n-a)-3(1-b)}H_{2a-3b} where n≦30, 0≦a≦n and b=0 or 1; and
aryl or heteroaryl having from 2 to 30 carbon atoms and their alkyl-, aryl-, heteroaryl-, cycloalkyl-, halogen-, hydroxy-, amino-, carboxy-, formyl-, —O—, —CO— or —CO—O-substituted components, for example phenyl, 2-methylphenyl (2-tolyl), 3-methylphenyl (3-tolyl), 4-methylphenyl, 2-ethylphenyl, 3-ethylphenyl, 4-ethylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 4-phenylphenyl, 1-naphthyl, 2-naphthyl, 1-pyrrolyl, 2-pyrrolyl, 3-pyrrolyl, 2-pyridinyl, 3-pyridinyl, 4-pyridinyl or C₆F₍₅₋ₐ₎Hₐ where 0≦a≦5.

When the anion is a tetra-substituted borate (Va) [BR^{a}R^{b}R^{c}R^{d}]⁻, then all four radicals R^{a} to R^{d} in this are preferably identical and are preferably fluorine, trifluoromethyl, pentafluoroethyl, phenyl, 3,5-bis(trifluoromethyl)phenyl. Particularly preferred tetrasubstituted borates (Va) are tetrafluoroborate, tetraphenylborate and tetra[3,5-bis(trifluoromethyl)phenyl]borate.

When the anion is an organic sulfonate (Vb) [R^{e}—SO₃]⁻, then the radical R^{e} is preferably methyl, trifluoromethyl, pentafluoroethyl, p-tolyl or C₉F₁₉. Particularly preferred organic sulfonates (Vb) are trifluoromethanesulfonate (triflate), methanesulfonate, p-toluenesulfonate, nonadecafluorononanesulfonate (nonaflate), dimethylene glycol monomethyl ether sulfate and octylsulfate.

When the anion is a carboxylate (Vc) [R^{f}—COO]⁻, then the radical R^{f} is preferably hydrogen, trifluoromethyl, pentafluoroethyl, phenyl, hydroxyphenylmethyl, trichloromethyl, dichloromethyl, chloromethyl, trifluoromethyl, difluoromethyl, fluoromethyl, ethenyl (vinyl), 2-propenyl, —CH═CH—COO⁻, cis-8-heptadecenyl, —CH₂—C(OH)(COOH)—CH₂—COO⁻ or unbranched or branched C₁-C₁₈-alkyl, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, heptadecyl. Particularly preferred carboxylates (Vc) are formate, acetate, propionate, butyrate, valerate, benzoate, mandelate, trichloroacetate, dichloroacetate, chloroacetate, trifluoroacetate, difluoroacetate, fluoroacetate.

When the anion is a (fluoroalkyl)fluorophosphate (Vd) [PFₓ(C_{y}F_{2y+1-z}H_{z})₆₋ₓ]⁻, then z is preferably 0. Particular preference is given to (fluoroalkyl)fluorophosphates (Vd), in which z=0, x=3 and 1≦y≦4, specifically [PF₃(CF₃)₃]⁻, [PF₃(C₂F₅)₃]⁻, [PF₃(C₃F₇)₃]⁻ and [PF₃(C₄F₇)₃]⁻.

When the anion is an imide (Ve) [R^{g}—SO₂—N—SO₂—R^{h}]⁻, (Vf) [Rⁱ—SO₂—N—CO—R^{j}]⁻ or (Vg) [R^{k}—CO—N—CO—R^{l}]⁻, then the radicals R^{g} to R^{l} are each preferably, independently of one another, trifluoromethyl, pentafluoroethyl, phenyl, trichloromethyl, dichloromethyl, chloromethyl, trifluoromethyl, difluoromethyl, fluoromethyl or unbranched or branched C₁-C₁₂-alkyl, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl or dodecyl. Particularly preferred imides (Ve), (Vf) and (Vg) are [F₃C—SO₂—N—SO₂—CF₃]⁻(bis(trifluoromethylsulfonyl)imide), [F₅C₂—SO₂—N—SO₂—C₂F₅]⁻(bis(pentafluoroethylsulfonyl)imide), [F₃C—SO₂—N—CO—CF₃]⁻, [F₃C—CO—N—CO—CF₃]⁻ and those in which the radicals R^{g} to R^{l} are each, independently of one another, methyl, ethyl, propyl, butyl, phenyl, trichloromethyl, dichloromethyl, chloromethyl, trifluoromethyl, difluoromethyl or fluoromethyl.

When the anion is a methide (Vh) 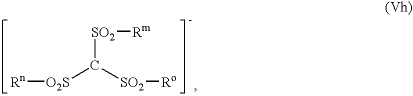
then the radicals R^{m} to R^{o} are each preferably, independently of one another, trifluoromethyl, pentafluoroethyl, phenyl, trichloromethyl, dichloromethyl, chloromethyl, trifluoromethyl, difluoromethyl, fluoromethyl or unbranched or branched C₁-C₁₂-alkyl, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl or dodecyl. Particularly preferred methides (Vh) are [(F₃C—SO₂)₃C]⁻ (tris(trifluoromethylsulfonyl)methide), [(F₅C₂—SO₂)₃C]⁻ (bis(pentafluoroethylsulfonyl)methide) and those in which the radicals R^{m} to R^{o} are each, independently of one another, methyl, ethyl, propyl, butyl, phenyl, trichloromethyl, dichloromethyl, chloromethyl, trifluoromethyl, difluoromethyl or fluoromethyl.

When the anion is an organic sulfate (Vi) [R^{p}O—SO₃]⁻, then the radical R^{p} is preferably a branched or unbranched C₁-C₃₀-alkyl radical. Particularly preferred organic sulfates (Vi) are methylsulfate, ethylsulfate, propylsulfate, butylsulfate, pentylsulfate, hexylsulfate, heptylsulfate or octylsulfate.

When the anion is a halometalate (Vj) [M_{q}Halᵣ]^{s-}, then M is preferably aluminum, zinc, iron, cobalt, antimony or tin. Hal is preferably chlorine or bromine and very particularly preferably chlorine. q is preferably 1, 2 or 3 and r and s are determined by the stoichiometry and charge on the metal ion.

When the anion is a thiolate (Vn) [R^{s}S]⁻, then the radical R^{s} is preferably a branched or unbranched C₁-C₃₀-alkyl radical. Particularly preferred thiolates (Vn) are methylsulfide, ethylsulfide, n-propylsulfide, n-butylsulfide, n-pentylsulfide, n-hexylsulfide, n-heptylsulfide, n-octylsulfide or n-dodecylsulfide.

The imidazolium methylsulfite (I) of the invention is very particularly preferably used for preparing an imidazolium salt in which the partially or fully deprotonated anion is tetrafluoroborate, hexafluorophosphate, trifluoromethanesulfonate, methanesulfonate, formate, acetate, mandelate, nitrate, nitrite, trifluoroacetate, sulfate, hydrogensulfate, methylsulfate, ethylsulfate, propylsulfate, butylsulfate, pentylsulfate, hexylsulfate, heptylsulfate, octylsulfate, phosphate, dihydrogenphosphate, hydrogenphosphate, propionate, tetrachloroaluminate, Al₂Cl₇⁻, chlorozincate, chloroferrate, bis(trifluoromethylsulfonyl)imide, bis(pentafluoroethylsulfonyl)imide, tris(trifluoromethylsulfonyl)methide, bis(pentafluoroethylsulfonyl)methide, p-toluenesulfonate, bis[salicylato(2-)]borate, tetracarbonylcobaltate, dimethylene glycol monomethyl ether sulfate, octylsulfate, oleate, stearate, acrylate, methacrylate, maleate, hydrogencitrate, vinylphosphonate, bis(pentafluoroethyl)phosphinate, bis[oxalato(2-)]borate, bis[1,2-benzenediolato(2-)-O,O′]borate, dicyanamide, tris(pentafluoroethyl)trifluorophosphate, tris(heptafluoropropyl)trifluorophosphate, tetracyanoborate or chlorocobaltate.

The imidazolium methylsulfites (I) of the invention are important starting compounds for the preparation of ionic, 1,3-disubstituted imidazolium-based liquids. They can be prepared in a simple manner, without the use of toxic alkylating agents, using an alkylating agent which can readily be prepared industrially, is readily available and reactive, in high yield and high purity, with the high purity also being able to be achieved without complicated purification. The imidazolium methylsulfites (I) of the invention can be converted simply and flexibly into the desired ionic, 1,3-disubstituted imidazolium-based liquid having the desired anion, and this can likewise be prepared in high yield and high purity, even without a separate, complicated purification.

The particular advantage of the imidazolium methylsulfites (I) of the invention is that in their reaction with water or an inorganic or organic protic acid to prepare the desired ionic, 1,3-disubstituted imidazolium-based liquids, the methylsulfite anion can readily be removed completely with formation of volatile methanol (in the case of the reaction with water to form the hydrogensulfite) or with formation of volatile methanol and volatile sulfur dioxide (in the case of the reaction with an inorganic or organic protic acid).

The desired ionic liquids which can be prepared from the imidazolium methylsulfites (I) of the invention can thus be used without problems in the electronics industry.

### EXAMPLES

### Example 1

### N,N′-Butylmethylimidazolium methylsulfite

21.11 g (0.192 mol) of dimethyl sulfite together with 100 ml of toluene were placed in a reaction vessel and a solution of 23.8 g (0.192 mol) of N-butylimidazole in 25 ml of toluene was added. The solution was stirred at 60° C. for 15 hours. During the reaction, a second liquid phase formed due to the N,N′-butylmethylimidazolium methylsulfite formed. After stirring was stopped, the two phases separated. The lower phase comprising N,N′-butylmethylimidazolium methylsulfite was separated off and shaken twice with ethyl acetate. N,N′-Butylmethylimidazolium methylsulfite was subsequently dried at 40° C. under a reduced pressure of 0.3 kPa (3 mbar). The weight of product obtained was 37.5 g, corresponding to 83% of the theoretical total yield (N,N′-butylmethylimidazolium methylsulfite and N,N′-butylmethylimidazolium methanesulfonate).

The liquid product obtained was analyzed by NMR spectroscopy and identified as N,N′-butylmethylimidazolium methylsulfite:

[1H-NMR, 400 Mhz], D₂O.: 0.9 ppm (t-3H); 1.3 ppm (m-2H); 1.8 ppm (m-2H); 2.8 ppm (s-3H-CH₃SO₃⁻); 3.4 ppm (s-3H); 3.8 ppm (s-3H); 4.2 ppm (t-2H); 7.4 ppm (d-2H); 8.7 ppm (s-1H).

In a quantitative evaluation of the NMR spectrum, the ratio of signals 2.8 ppm (3H-methanesulfonate): 3.8 ppm (3H-methyl group on the imidazolium nitrogen) indicated that the proportion of methanesulfonate formed was below the detection limit. This is 3 mol %. The purity of the N,N′-butylmethylimidazolium methylsulfite was thus >97%.

### Example 2

### Synthesis of N,N′-butylmethylimidazolium acetate from N,N′-butylmethylimidazolium methylsulfite

49.3 g (0.21 mol) of N,N′-butylmethylimidazolium methylsulfite which had been prepared from batches according to example 1 were placed in a reaction vessel at room temperature and 13 g (0.21 mol) of acetic acid were slowly added dropwise while stirring. The reaction mixture was carefully placed under a reduced pressure of from 50 to 0.2 kPa (500 to 2 mbar) at from 40 to 65° C., with methanol formed being distilled off. After methanol formation and distillation were complete, the reaction mixture was heated to 140° C. and freed of sulfur dioxide under a reduced pressure of 0.3 kPa (3 mbar). The sulfur dioxide was collected in a cold trap. The yield of reaction product was 37.2 g, corresponding to 90% of the theoretical total yield.

The liquid product obtained was analyzed by NMR spectroscopy and identified as N,N′-butylmethylimidazolium acetate:

[1H-NMR, 400 Mhz], D₂O.: 0.9 ppm (t-3H); 1.3 ppm (m-2H); 1.8 ppm (m-2H); 1.9 ppm (s-3H CH₃COO⁻); 3.4 ppm (s-3H); 3.8 ppm (s-3H); 4.2 ppm (t-2H); 7.4 ppm (d-2H); 8.7 ppm (s-1H)

Example 2 shows that the desired ionic liquid N,N′-butylmethylimidazolium acetate can be prepared in high yield and high purity from the N,N′-butylmethylimidazolium methylsulfite according to the invention in a simple reaction with acetic acid.

1-13. (canceled)

14. An imidazolium methylsulfite of the general formula (I) 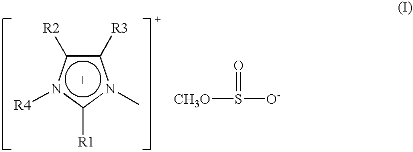
wherein
R¹, R³, and R³
is hydrogen, halogen, hydroxy, amino, aminocarbonyl, sulfo, cyano, nitro; C₁-C₂₀-alkyl, C₂-C₂₀-alkenyl, C₂-C₂₀-alkynyl, C₁-C₂₀-alkyloxy, C₂-C₂₀-alkenyloxy, C₂-C₂₀-alkynloxy, C₁-C₂₀-alkylcarbonyl, C₁-C₂₀-alkyloxycarbonyl, C₁-C₂₀-acyloxy, C₁-C₂₀-alkylaminocarbonyl, di(C₁-C₂₀-alkyl)aminocarbonyl, C₁-C₂₀-alkylamino, di(C₁-C₂₀-alkyl)amino, C₁-C₂₀-alkylsulfonyl, wherein said alkyl, alkenyl, and alkynyl substituents are optionally partially or fully halogenated and/or are optionally substituted with up to three of hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl and/or up to five CH₂ groups of said alkyl, alkenyl, and alkynyl substituents are optionally replaced with oxygen, wherein at least one carbon atom is present between any two oxygen atoms; C₃-C₈-cycloalkyl, wherein said cycloalkyl substituent is optionally partially or fully halogenated and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or up to three CH₂ groups of said alkyl, alkenyl and alkynyl substituents are optionally replaced with oxygen, wherein at least one carbon atom is present between any two oxygen atoms; phenyl, phenoxy, wherein said phenyl and phenoxy substituents these are optionally partially or fully halogenated and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl;
R⁴
is sulfo; C₁-C₂₀-alkyl, C₂-C₂₀-alkenyl, C₂-C₂₀-alkynyl, wherein said alkyl, alkenyl and alkynyl substituents are optionally partially or fully halogenated and/or are optionally substituted with up to three of hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully halogenated, and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to five CH₂ groups of said alkyl, alkenyl, and alkynyl substituents are optionally replaced with oxygen, wherein at least one carbon atom is present between any two oxygen atoms; C₃-C₈-cycloalkyl, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to three CH₂ groups of said cycloalkyl substituent are optionally replaced with oxygen, wherein at least one carbon atom is present between any two oxygen atoms; phenyl, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; or
R¹ together with R⁴, and/or R² together with R³, or R⁴ together with R² is alkylene of formula
—(CH₂)ₙ—
wherein n=4, 5 or 6, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully halogenated and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to two CH₂ groups of said alkylene are optionally replaced with oxygen or NR⁵, wherein at least one carbon atom is present between any two heteroatoms; or
—CH═CH—CH═CH—,
which is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents can are optionally partially or fully halogenated and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or up to two CH groups —CH═CH—CH═CH— are optionally replaced with NR⁵, wherein at least one carbon atom is present between any two heteroatoms, and wherein R⁵ is hydrogen, C₁-C₆-alkyl, C₁-C₆-haloalkyl or C₁-C₆-alkyloxy.

15. The imidazolium methylsulfite of claim 14, wherein R¹, R², and R³ are, independently of one another, hydrogen, fluorine, chlorine, aminocarbonyl, sulfo, cyano; C₁-C₂₀-alkyl, C₁-C₂₀-alkyloxy, C₁-C₂₀-alkylcarbonyl, C₁-C₂₀-alkyloxycarbonyl, C₁-C₂₀-acyloxy, C₁-C₂₀-alkylaminocarbonyl, di(C₁-C₂₀-alkyl)aminocarbonyl, C₁-C₂₀-alkylamino, di(C₁-C₂₀-alkyl)amino, wherein said alkyl substituents are optionally partially or fully fluorinated or chlorinated and/or are optionally substituted with up to three of hydroxy, sulfo, cyano, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully fluorinated or chlorinated and/or are optionally substituted with up to three of hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl; and/or up to five CH₂ groups of said alkyl substituents are optionally replaced with oxygen, wherein at least one carbon atom is present between any two oxygen atoms; C₃-C₈-cycloalkyl, which is optionally partially or fully fluorinated or chlorinated, and/or is optionally substituted with up to three of hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl, and/or up to three CH₂ groups of said cycloalkyl substituents are optionally replaced with oxygen, wherein at least one carbon atom is present between any two oxygen atoms; phenyl, and phenoxy, wherein said phenyl and phenoxy substituents are optionally partially or fully fluorinated or chlorinated, and/or are optionally substituted with up to three of hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl.

16. The imidazolium methylsulfite of claim 15, wherein R¹, R³, and R³ are, independently of one another, hydrogen, fluorine, chlorine; C₁-C₆-alkyl, C₁-C₆-alkyloxy, C₅-C₆-cycloalkyl, wherein said alkyl and cycloalkyl substituents are optionally partially or fully fluorinated or chlorinated, and/or up to two of the CH₂ groups of said alkyl and cycloalkyl substituents are optionally replaced with oxygen, wherein at least one carbon atom is present between any two oxygen atoms; and phenyl, which is optionally partially or fully fluorinated or chlorinated, and/or optionally substituted with up to three of C₁-C₆-alkyl, C₁-C₆-haloalkyl, C₁-C₆-alkyloxy.

17. The imidazolium methylsulfite of claim 16, wherein R¹, R³, and R³ are, independently of one another, hydrogen, methyl, ethyl, 1-propyl, 1-butyl, 1-pentyl, 1-hexyl, 1-octyl, 1-decyl, 1-dodecyl, 1-pentadecyl.

18. The imidazolium methylsulfite of claim 14, wherein R⁴ is sulfo; C₁-C₂₀-alkyl, which is optionally partially or fully fluorinated or chlorinated, and/or optionally substituted with up to three of hydroxy, sulfo, cyano, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy groups are optionally partially or fully fluorinated or chlorinated and/or are optionally substituted with up to three of hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl; and/or up to five CH₂ groups of said alkyl substituents are replaced with oxygen, wherein at least one carbon atom is present between any two oxygen atoms; and C₃-C₈-cycloalkyl, which is optionally partially or fully fluorinated or chlorinated, and/or optionally substituted with up to three of hydroxy, sulfo, cyano, amino, aminocarbonyl, C₁-C₆-alkyl, C₁-C₆-haloalkyl, C₁-C₆-alkyloxy, C₁-C₆-alkyloxycarbonyl, C₁-C₆-alkylcarbonyl, and/or up to three CH₂ groups of said C₃-C₈-cycloalkyl substituents are optionally replaced with oxygen, wherein at least one carbon atom is present between any two oxygen atoms.

19. The imidazolium methylsulfite of claim 18, wherein R⁴ is sulfo, C₁-C₆-alkyl, and C₅-C₆-cycloalkyl, where said alkyl and cycloalkyl substituents are optionally partially or fully fluorinated or chlorinated, and/or up to two CH₂ groups of said alkyl and cycloalkyl substituents are optionally replaced with oxygen, wherein at least one carbon atom is present between any two oxygen atoms.

20. The imidazolium methylsulfite of claim 19, wherein R⁴ is methyl, ethyl, 1-propyl, 1-butyl, 1-pentyl, 1-hexyl, 1-octyl, 1-decyl, 1-dodecyl, or 1-pentadecyl.

21. The imidazolium methylsulfite of claim 14, wherein said imidazolium methylsulfite is selected from the group consisting of N,N′-Dimethylimidazolium methylsulfite, N,N′-ethylmethylimidazolium methylsulfite, N,N′-(1-propyl)methylimidazolium methylsulfite, N,N′-(1-butyl)methylimidazolium methylsulfite, N,N′-(1-pentyl)methylimidazolium methylsulfite, N,N′-(1-hexyl)methylimidazolium methylsulfite, N,N′-(1-octyl)methylimidazolium methylsulfite, N,N′-(1-decyl)methylimidazolium methylsulfite, N,N′-(1-dodecyl)methylimidazolium methylsulfite, and N,N′-(1-pentadecyl)methylimidazolium methylsulfite.

22. A process for preparing imidazolium methylsulfites of claim 14, comprising the step of reacting the an imidazole of the general formula (II) 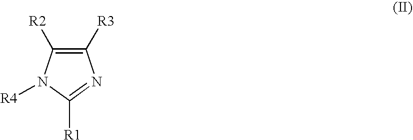
with dimethyl sulfite, wherein
R¹, R³, and R³
is hydrogen, halogen, hydroxy, amino, aminocarbonyl, sulfo, cyano, nitro; C₁-C₂₀-alkyl, C₂-C₂₀-alkenyl, C₂-C₂₀-alkynyl, C₁-C₂₀-alkyloxy, C₂-C₂₀-alkenyloxy, C₂-C₂₀-alkynloxy, C₁-C₂₀-alkylcarbonyl, C₁-C₂₀-alkyloxycarbonyl, C₁-C₂₀-acyloxy, C₁-C₂₀-alkylaminocarbonyl, di(C₁-C₂₀-alkyl)aminocarbonyl, C₁-C₂₀-alkylamino, di(C₁-C₂₀-alkyl)amino, C₁-C₂₀-alkylsulfonyl, wherein said alkyl, alkenyl, and alkynyl substituents are optionally partially or fully halogenated and/or are optionally substituted with up to three of hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl and/or up to five CH₂ groups of said alkyl, alkenyl, and alkynyl substituents are optionally replaced with oxygen, wherein at least one carbon atom is present between any two oxygen atoms; C₃-C₈-cycloalkyl, wherein said cycloalkyl substituent is optionally partially or fully halogenated and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or up to three CH₂ groups of said alkyl, alkenyl and alkynyl substituents are optionally replaced with oxygen, wherein at least one carbon atom is present between any two oxygen atoms; phenyl, phenoxy, wherein said phenyl and phenoxy substituents these are optionally partially or fully halogenated and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl;
R⁴
is sulfo; C₁-C₂₀-alkyl, C₂-C₂₀-alkenyl, C₂-C₂₀-alkynyl, wherein said alkyl, alkenyl and alkynyl substituents are optionally partially or fully halogenated and/or are optionally substituted with up to three of hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully halogenated, and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to five CH₂ groups of said alkyl, alkenyl, and alkynyl substituents are optionally replaced with oxygen, wherein at least one carbon atom is present between any two oxygen atoms;
C₃-C₈-cycloalkyl, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to three CH₂ groups of said cycloalkyl substituent are optionally replaced with oxygen, wherein at least one carbon atom is present between any two oxygen atoms; phenyl, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; or
R¹ together with R⁴, and/or R² together with R³, or R⁴ together with R² is alkylene of formula
—(CH₂)ₙ—
wherein n=4, 5 or 6, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully halogenated and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to two CH₂ groups of said alkylene are optionally replaced with oxygen or NR⁵, wherein at least one carbon atom is present between any two heteroatoms; or
—CH═CH—CH═CH—,
which is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents can are optionally partially or fully halogenated and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or up to two CH groups —CH═CH—CH═CH— are optionally replaced with NR⁵, wherein at least one carbon atom is present between any two heteroatoms, and wherein R⁵ is hydrogen, C₁-C₆-alkyl, C₁-C₆-haloalkyl or C₁-C₆-alkyloxy.

23. The process according to claim 22, wherein said reaction step is carried out
(i) in the presence of a solvent selected from the group consisting of aromatic hydrocarbons having from 6 to 10 carbon atoms, symmetrical or unsymmetrical dialkyl ethers having a total of from 5 to 10 carbon atoms, cycloalkanes having from 5 to 8 carbon atoms, and C₅-C₁₀-alkanes; and
(ii) at a temperature of from 10 to 100° C.

24. A process for preparing an imidazolium hydrogensulfite, comprising the step of reacting the an imidazolium methylsulfite of the general formula (I) 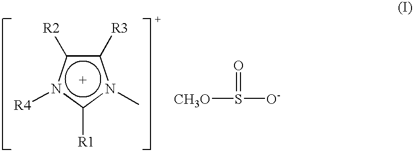
with water, wherein
R¹, R³, and R³
is hydrogen, halogen, hydroxy, amino, aminocarbonyl, sulfo, cyano, nitro; C₁-C₂₀-alkyl, C₂-C₂₀-alkenyl, C₂-C₂₀-alkynyl, C₁-C₂₀-alkyloxy, C₂-C₂₀-alkenyloxy, C₂-C₂₀-alkynloxy, C₁-C₂₀-alkylcarbonyl, C₁-C₂₀-alkyloxycarbonyl, C₁-C₂₀-acyloxy, C₁-C₂₀-alkylaminocarbonyl, di(C₁-C₂₀-alkyl)aminocarbonyl, C₁-C₂₀-alkylamino, di(C₁-C₂₀-alkyl)amino, C₁-C₂₀-alkylsulfonyl, wherein said alkyl, alkenyl, and alkynyl substituents are optionally partially or fully halogenated and/or are optionally substituted with up to three of hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl and/or up to five CH₂ groups of said alkyl, alkenyl, and alkynyl substituents are optionally replaced with oxygen, wherein at least one carbon atom is present between any two oxygen atoms; C₃-C₈-cycloalkyl, wherein said cycloalkyl substituent is optionally partially or fully halogenated and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or up to three CH₂ groups of said alkyl, alkenyl and alkynyl substituents are optionally replaced with oxygen, wherein at least one carbon atom is present between any two oxygen atoms; phenyl, phenoxy, wherein said phenyl and phenoxy substituents these are optionally partially or fully halogenated and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl;
R⁴
is sulfo; C₁-C₂₀-alkyl, C₂-C₂₀-alkenyl, C₂-C₂₀-alkynyl, wherein said alkyl, alkenyl and alkynyl substituents are optionally partially or fully halogenated and/or are optionally substituted with up to three of hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully halogenated, and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to five CH₂ groups of said alkyl, alkenyl, and alkynyl substituents are optionally replaced with oxygen, wherein at least one carbon atom is present between any two oxygen atoms; C₃-C₈-cycloalkyl, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to three CH₂ groups of said cycloalkyl substituent are optionally replaced with oxygen, wherein at least one carbon atom is present between any two oxygen atoms; phenyl, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; or
R¹ together with R⁴, and/or R² together with R³, or R⁴ together with R² is alkylene of formula
—(CH₂)ₙ—
wherein n=4, 5 or 6, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully halogenated and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to two CH₂ groups of said alkylene are optionally replaced with oxygen or NR⁵, wherein at least one carbon atom is present between any two heteroatoms; or
—CH═CH—CH═CH—,
which is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents can are optionally partially or fully halogenated and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or up to two CH groups —CH═CH—CH═CH— are optionally replaced with NR⁵, wherein at least one carbon atom is present between any two heteroatoms, and wherein R⁵ is hydrogen, C₁-C₆-alkyl, C₁-C₆-haloalkyl or C₁-C₆-alkyloxy.

25. A process for preparing an imidazolium salt of an inorganic or organic protic acid comprising the step of reacting the an imidazolium methylsulfite of the general formula (I) 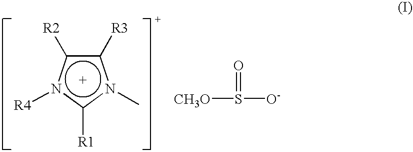
with an inorganic or organic protic acid having a pKₐ of from 1.8 to 14, measured at 25° C. in aqueous solution, wherein
R¹, R³, and R³
is hydrogen, halogen, hydroxy, amino, aminocarbonyl, sulfo, cyano, nitro; C₁-C₂₀-alkyl, C₂-C₂₀-alkenyl, C₂-C₂₀-alkynyl, C₁-C₂₀-alkyloxy, C₂-C₂₀-alkenyloxy, C₂-C₂₀-alkynloxy, C₁-C₂₀-alkylcarbonyl, C₁-C₂₀-alkyloxycarbonyl, C₁-C₂₀-acyloxy, C₁-C₂₀-alkylaminocarbonyl, di(C₁-C₂₀-alkyl)aminocarbonyl, C₁-C₂₀-alkylamino, di(C₁-C₂₀-alkyl)amino, C₁-C₂₀-alkylsulfonyl, wherein said alkyl, alkenyl, and alkynyl substituents are optionally partially or fully halogenated and/or are optionally substituted with up to three of hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, where the C₃-C₈-cycloalkyl, phenyl and phenoxy groups can in turn be partially or fully halogenated and/or can bear from one to three of the following groups: hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl and/or up to five CH₂ groups of said alkyl, alkenyl, and alkynyl substituents are optionally replaced with oxygen, wherein at least one carbon atom is present between any two oxygen atoms; C₃-C₈-cycloalkyl, wherein said cycloalkyl substituent is optionally partially or fully halogenated and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or up to three CH₂ groups of said alkyl, alkenyl and alkynyl substituents are optionally replaced with oxygen, wherein at least one carbon atom is present between any two oxygen atoms; phenyl, phenoxy, wherein said phenyl and phenoxy substituents these are optionally partially or fully halogenated and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl;
R⁴
is sulfo; C₁-C₂₀-alkyl, C₂-C₂₀-alkenyl, C₂-C₂₀-alkynyl, wherein said alkyl, alkenyl and alkynyl substituents are optionally partially or fully halogenated and/or are optionally substituted with up to three of hydroxy, sulfo, cyano, nitro, amino, aminocarbonyl, di(C₁-C₆-alkyl)amino, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully halogenated, and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to five CH₂ groups of said alkyl, alkenyl, and alkynyl substituents are optionally replaced with oxygen, wherein at least one carbon atom is present between any two oxygen atoms; C₃-C₈-cycloalkyl, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to three CH₂ groups of said cycloalkyl substituent are optionally replaced with oxygen, wherein at least one carbon atom is present between any two oxygen atoms; phenyl, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; or
R¹ together with R⁴, and/or R² together with R³, or R⁴ together with R² is alkylene of formula
—(CH₂)ₙ—
wherein n=4, 5 or 6, which is optionally partially or fully halogenated, and/or is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents are optionally partially or fully halogenated and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl, and/or up to two CH₂ groups of said alkylene are optionally replaced with oxygen or NR⁵, wherein at least one carbon atom is present between any two heteroatoms; or
—CH═CH—CH═CH—,
which is optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkylcarbonyl, C₁-C₆-alkyloxycarbonyl, C₃-C₈-cycloalkyl, phenyl, phenoxy, wherein said C₃-C₈-cycloalkyl, phenyl and phenoxy substituents can are optionally partially or fully halogenated and/or are optionally substituted with up to three of hydroxy, sulfo, cyano, nitro, amino, aminocarbonyl, C₁-C₆-alkyl, C₁-C₆-haloalkyl, C₁-C₆-alkyloxy, C₁-C₆-haloalkyloxy, C₁-C₆-alkyloxycarbonyl, C₁-C₆-alkylcarbonyl; and/or up to two CH groups —CH═CH—CH═CH— are optionally replaced with NR⁵, wherein at least one carbon atom is present between any two heteroatoms, and wherein R⁵ is hydrogen, C₁-C₆-alkyl, C₁-C₆-haloalkyl or C₁-C₆-alkyloxy.

26. The process according to claim 25, wherein said partially or fully deprotonated anion is tetrafluoroborate, hexafluorophosphate, trifluoromethanesulfonate, methanesulfonate, formate, acetate, mandelate, nitrate, nitrite, trifluoroacetate, sulfate, hydrogensulfate, methylsulfate, ethylsulfate, propylsulfate, butylsulfate, pentylsulfate, hexylsulfate, heptylsulfate, octylsulfate, phosphate, dihydrogenphosphate, hydrogenphosphate, propionate, tetrachloroaluminate, Al₂Cl₇⁻, chlorozincate, chloroferrate, bis(trifluoroethylsulfonyl)imide, bis(pentafluoroethylsulfonyl)imide, tris(trifluoromethylsulfonyl)ethide, bis(pentafluoroethylsulfonyl)methide, p-tolylsulfonate, bis[salicylato(2-)]borate, tetracarbonylcobaltate, dimethylene glycol monomethyl ether sulfate, octylsulfate, oleate, stearate, acrylate, methacrylate, maleate, hydrogencitrate, vinylphosphonate, bis(pentafluoroethyl)phosphinate, bis[oxalato(2-)]borate, bis[1,2-benzenediolato(2-)-O,O′]borate, dicyanamide, tris(pentafluoroethyl)trifluorophosphate, tris(heptafluoropropyl)trifluorophosphate, tetracyanoborate or chlorocobaltate is prepared.
